(19) United States
(12) Patent Application Publication
(10) Pub. No.: US 2026/0174335 A1
(43) Pub. Date: Jun. 25, 2026

(54) FLUORESCENCE IMAGING SYSTEM FOR WOUNDS

(71) Applicant: eKare Inc., Fairfax, VA (US)

(72) Inventors: Necip Gurler, Fairfax, VA (US); Patrick Cheng, Fairfax, VA (US)

(21) Appl. No.: 19/544,916

(22) Filed: Feb. 19, 2026

Related U.S. Application Data
(63) Continuation of application No. PCT/US2024/043221, filed on Aug. 21, 2024.
(60) Provisional application No. 63/520,709, filed on Aug. 21, 2023.

(51) Int. Cl. A61B 5/00; G06T 7/00

(52) U.S. Cl. CPC A61B 5/0059; G06T 7/0012

(57) ABSTRACT

Provided herein are methods, apparatuses, computer program products, and systems for fluorescence imaging. One method can include activating an ultraviolet (UV) light source to transmit UV light at a predetermined wavelength that is below a band of human visual perception to illuminate a region of a human body; generating a fluorescent image of the region of the human body based on fluorescent signals that are emitted from the region of the human body in response to illumination using the UV light and are captured by the camera device; and performing one or more actions using the fluorescent image of the region of the human body.

### CROSS-REFERENCE TO RELATED APPLICATIONS

This patent application is a by-pass continuation and claims priority to PCT Application No. PCT/US2024/043221, filed on Aug. 21, 2024, which claims priority to and the benefit of U.S. Provisional Patent Application No. 63/520,709 , filed on Aug. 21, 2023, which is incorporated herein by reference in its entirety.

### BACKGROUND

Managing wound care presents a significant clinical challenge, especially within an aging population. Both healing and chronic non-healing wounds are closely associated with a range of biological tissue changes, with bacterial infection being the most critical.

A certain proportion of wound infections may not be immediately clinically apparent, creating a concealed but growing burden in wound care management. This situation subsequently escalates health care costs. Furthermore, wound infection can lead to serious complications such as delayed healing, amputation, and increased mortality in some cases. Thus, early diagnosis of bacterial presence in wounds is critical for prevention and control of the spread of infections.

The current standard procedure for wound assessment involves direct visual inspection of the wound site under white light, supplemented by the unspecific collection of bacterial swabs and tissue biopsies.

Some techniques use one or more red, green, blue (RGB) cameras to capture a color image under the visible spectrum. While these color images depict what human eyes can see, the challenge lies in identifying bacterial infection in the wounds using only these images. Consequently, the effective prevention and control of infections heavily rely on the rapid and accurate diagnosis of bacterial presence in wounds.

To address the aforementioned complications in wound care management, traditional fluorescence imaging has been applied, for example, using blue light with a wavelength of 405 nm, which is considered visible light. After illuminating the specimen using blue light, the fluorophore of the specimen can emit fluorescence signals. To enhance the visualization, the illumination light can be blocked, for example, by using a bandpass filter. However, interpreting the result to determine the presence or absence of bacterial growth remains a significant challenge.

### SUMMARY

The disclosed systems, methods, and techniques generally relate to fluorescence imaging for bacteria detection in wounds. Specifically, the systems and techniques describe a novel, smartphone-based full visible spectrum fluorescence imaging system for non-invasive bacterial assessment at the point-of-care.

In general, one innovative aspect of the subject matter described in this specification can be embodied in systems that include: an ultraviolet (UV) light source configured to illuminate a region of a human body using UV light at a predetermined wavelength; and a computing device to which the UV light source is attached, wherein the computing device is arranged in communication with the UV light source and configured to activate the UV light source using a software program installed on the computing device, the computing device including: a camera device configured to detect fluorescent signals emitted from the region of the human body in response to illumination using the UV light at the predetermined wavelength that is below a band of human visual perception, a screen, a memory device, and one or more processors coupled to the camera device, the screen, and the memory device, wherein the one or more processors are configured to control the UV light source, the camera device, the screen, and the memory device to act in tandem such that, in response to illumination using the UV light at the predetermined wavelength that is below the band of human visual perception, the camera device captures fluorescent signals, and the screen provides a fluorescent image of the region of the human body based on the fluorescent signals. Other embodiments of this aspect include corresponding methods, apparatus, and computer programs recorded on one or more computer storage devices, each configured to perform operations of the one or more processors of the systems. For one or more computer programs to be configured to perform particular operations or actions means that the one or more programs include instructions that, when executed by data processing apparatus, cause the apparatus to perform the operations or actions.

The foregoing and other embodiments can each optionally include one or more of the following features, alone or in combination. In particular, one embodiment includes all the following features in combination. Once activated, the UV light source illuminates the region of the human body in a dark room. Once activated, the UV light source illuminates the region of the human body in a room with light. The fluorescent signals include all fluorescent signals emitted from the region of the human body that are within a receptive range of the camera device. The predetermined wavelength is shorter than 400 nanometers (nm). The predetermined wavelength is about 365 nm. The predetermined wavelength is characterized by a center wavelength along with a width of wavelength, wherein the center wavelength is shorter than 380 nm, and wherein the width of wavelength is less than 20 nm. The one or more processors are configured to control UV light source such that the UV light source is modulated when illuminating the region of the human body. The UV light source is modulated according to at least one of: a pulse width modulation, a pulse intensity modulation, or a frequency modulation. The one or more processors are configured to analyze the fluorescent image by: segmenting at least one region in the fluorescent image with likely bacterial presence using a machine learning model to discern characteristic features of bacterial induced fluorescence.

The one or more processors are configured to process sequential fluorescent images of the region of the human body using a machine learning model, to track a change in the sequential fluorescent images of the region of the human body and to predict a healing trajectory of the region of the human body. The one or more processors of the computing device are configured to generate, based on the fluorescent image, an enhanced fluorescent image that has better interpretability than that of the fluorescent image. The one or more processors of the computing device are configured to: generate Hue values of the fluorescent image; and generate the enhanced fluorescent image based on the Hue values of the fluorescent image. The one or more processors of the computing device are configured to: process the fluorescent image using a Generative Adversarial Network (GAN) trained on a dataset of fluorescent images to generate the enhanced fluorescent image. The one or more processors of the computing device are configured to process the fluorescent image using a Generative Adversarial Network (GAN) to generate a prediction of at least one future state of the region of the human body under at least one treatment scenario. The one or more processors of the computing device are configured to: provide, based on the at least one predicted future state, a visual simulation of how the region of the human body evolves under the least one treatment scenario. The fluorescent image is a fluorescent image of a wound area of the human body.

The one or more processors of the computing device are configured to perform operations including: activating, by the one or more computer processors, an ultraviolet (UV) light source to transmit UV light at a predetermined wavelength that is below a band of human visual perception to illuminate a region of a human body; generating, by the one or more computer processors, a fluorescent image of the region of the human body based on fluorescent signals that are emitted from the region of the human body in response to illumination using the UV light and are captured by the camera device; and performing, by the computer one or more processors, one or more actions using the fluorescent image of the region of the human body. Performing the one or more actions using the fluorescent image includes displaying the fluorescent image of the region of the human body on a screen. Performing the one or more actions using the fluorescent image includes generating a diagnostic result of bacteria presence in the region of the human body based on the fluorescent image. The diagnostic result includes a score indicating a likelihood that there are bacteria in the region of the human body. The diagnostic result includes data indicating one or more regions in the fluorescent image that likely have bacteria. The diagnostic result includes a level of bacterial infection in the region of the human body. Performing the one or more actions using the fluorescent image includes processing the fluorescent image using a machine learning model to generate a bacteria species classification of the bacteria presence in the region of the human body. Performing the one or more actions using the fluorescent image includes processing the fluorescent image using a machine learning model to generate a quantification of one or more bacteria species of the bacteria presence in the region of the human body. The operations include capturing the fluorescent image of the region of the human body and a natural image of the region of the human body almost at a same time by switching on and off the UV light, causing the fluorescent image and the natural image being well aligned with each other. The operations further include displaying both the fluorescent image and the natural image of the region of the human body. The operations further include displaying a slider that allows a user to slide between the fluorescent image and the natural image that overlays on top of each other. Displaying the fluorescent image includes displaying an enhanced fluorescent image generated based on the fluorescent image, wherein the enhanced fluorescent image has better interpretability than that of the fluorescent image. The one or more processors include one or more processors of a computing device that includes the camera device. The one or more processors further include one or more processors of a cloud computer that are located remotely from the computing device and are connected with the computing device through a network. Performing the one or more actions using the fluorescent image includes: decomposing the fluorescent image into wavelength components; and performing fluorescence spectroscopy analysis using the wavelength components of the fluorescent image. The operations further include determining one or more environmental parameters using one or more sensors; and controlling parameters of the camera device, parameters of the UV light source, or both, based on the one or more environmental parameters to maintain consistent signal strength of the UV light that is used for capturing consecutive fluorescent images for a region of interest (ROI). Performing the one or more actions using the fluorescent image includes: searching a database of previously captured images to retrieve one or more images that are similar to the fluorescent image; and generating a diagnostic result of the region of the human body based on the fluorescent image and diagnostic result of the one or more images that are similar to the fluorescent image.

In general, another innovative aspect of the subject matter described in this specification can be embodied in methods that include the actions of activating, by the one or more computer processors, an ultraviolet (UV) light source to transmit UV light at a predetermined wavelength that is below a band of human visual perception to illuminate a region of a human body; generating, by the one or more computer processors, a fluorescent image of the region of the human body based on fluorescent signals that are emitted from the region of the human body in response to illumination using the UV light and are captured by the camera device; and performing, by the computer one or more processors, one or more actions using the fluorescent image of the region of the human body. Other embodiments of this aspect include corresponding computer systems, apparatus, and computer programs recorded on one or more computer storage devices, each configured to perform the actions of the methods. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

The foregoing and other embodiments can each optionally include one or more of the following features, alone or in combination. In particular, one embodiment includes all the following features in combination. Performing the one or more actions using the fluorescent image includes displaying the fluorescent image of the region of the human body on a screen. Performing the one or more actions using the fluorescent image includes generating a diagnostic result of bacteria presence in the region of the human body based on the fluorescent image. The diagnostic result includes a score indicating a likelihood that there are bacteria in the region of the human body. The diagnostic result includes data indicating one or more regions in the fluorescent image that likely have bacteria. The diagnostic result includes a level of bacterial infection in the region of the human body. Performing the one or more actions using the fluorescent image includes processing the fluorescent image using a machine learning model to generate a bacteria species classification of the bacteria presence in the region of the human body. Performing the one or more actions using the fluorescent image includes processing the fluorescent image using a machine learning model to generate a quantification of one or more bacteria species of the bacteria presence in the region of the human body. The actions include capturing the fluorescent image of the region of the human body and a natural image of the region of the human body almost at a same time by switching on and off the UV light, causing the fluorescent image and the natural image being well aligned with each other. The actions include displaying both the fluorescent image and the natural image of the region of the human body. The actions include displaying a slider that allows a user to slide between the fluorescent image and the natural image that overlays on top of each other. Displaying the fluorescent image includes displaying an enhanced fluorescent image generated based on the fluorescent image, wherein the enhanced fluorescent image has better interpretability than that of the fluorescent image.

The one or more processors include one or more processors of a computing device that includes the camera device. The one or more processors further include one or more processors of a cloud computer that are located remotely from the computing device and are connected with the computing device through a network. Performing the one or more actions using the fluorescent image includes: decomposing the fluorescent image into wavelength components; and performing fluorescence spectroscopy analysis using the wavelength components of the fluorescent image. The actions include determining one or more environmental parameters using one or more sensors; and controlling parameters of the camera device, parameters of the UV light source, or both, based on the one or more environmental parameters to maintain consistent signal strength of the UV light that is used for capturing consecutive fluorescent images for a region of interest (ROI). Performing the one or more actions using the fluorescent image includes: searching a database of previously captured images to retrieve one or more images that are similar to the fluorescent image; and generating a diagnostic result of the region of the human body based on the fluorescent image and diagnostic result of the one or more images that are similar to the fluorescent image.

The actions include controlling UV light source such that the UV light source is modulated when illuminating the region of the human body. The actions include analyzing the fluorescent image by segmenting at least one region in the fluorescent image with likely bacterial presence using a machine learning model to discern characteristic features of bacterial induced fluorescence. The actions include processing sequential fluorescent images of the region of the human body using a machine learning model, to track a change in the sequential fluorescent images of the region of the human body and to predict a healing trajectory of the region of the human body. The actions include generating, based on the fluorescent image, an enhanced fluorescent image that has better interpretability than that of the fluorescent image. The actions include generating Hue values of the fluorescent image; and generating the enhanced fluorescent image based on the Hue values of the fluorescent image. The actions include processing the fluorescent image using a Generative Adversarial Network (GAN) trained on a dataset of fluorescent images to generate the enhanced fluorescent image. The actions include processing the fluorescent image using a Generative Adversarial Network (GAN) to generate a prediction of at least one future state of the region of the human body under at least one treatment scenario. The actions include providing, based on the at least one predicted future state, a visual simulation of how the region of the human body evolves under the least one treatment scenario.

Particular embodiments of the subject matter described in this specification can be implemented so as to realize one or more of the following advantages. The systems and techniques can perform fluorescence imaging by attaching an ultraviolet (UV) light source to a mobile device, without requiring an additional dedicated imaging device. Instead of requiring a dark room or dark drape, the systems and techniques can perform the fluorescence imaging under ambient light. Rather than being limited to a limited set of colors, e.g., by using a filter in front of a camera of the imaging device and allowing certain colors to pass the filter (e.g., green, cyan, and red colors only) which may result in interpretation and saturation issues, the systems and techniques can obtain full visible spectrum fluorescence imaging, providing higher color resolution and improved image quality for human or automatic diagnosis of bacteria presence in wounds. The systems and techniques can image the full visible fluorescence spectrum with enhanced diagnostic clarity in regions with bacterial growth, as well as the surrounding tissues. The systems and techniques can significantly improve the accuracy of wound diagnosis and treatment efficiency at the point-of-care, particularly in applications such as bacterial load assessment or fluorescence imaging-guided debridement.

In some implementations, the systems and techniques can provide improved differentiation of bacteria species in wounds. In some implementations, besides qualitative measurements, the systems and techniques can provide quantitative measurements of the wounds based on quantification of the UV signal strength through controlling the variables of the system, including parameters of ambient light sensor, camera exposure, and other parameters of the camera, strength of the UV, etc. With the quantitative measurements of the wounds, the systems and techniques can automatically determine whether the wound has bacterial infection.

In some implementations, the systems and techniques can capture a fluorescent image and a colored image taken from the range of visible wavelengths (referred to as “a natural color image” or “a normal image” thereafter) almost at the same time. Thus, the two images can be well aligned with each other, improving the convenience for future (automatic) comparison and analysis of the images.

In some implementations, the systems and techniques can perform automatic analysis of the fluorescent image using machine learning and artificial intelligence and can generate a report showing the diagnostic result of the wound, e.g., indicating a bacteria infected area of the wound. Specifically, in some implementations, a convolutional neural network (CNN)—a specialized type of neural network for processing visual data—can be used. Using the CNN features, the implementations can decipher the intricate variations in the fluorescence and color images, isolating indicative markers of infection. For example, distinct fluorescence traits associated with different types of bacteria can be recognized. This could streamline the optimization of treatment plans, by tailoring them to the specific type of infection identified.

The details of one or more embodiments of the subject matter described in this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 shows an example environment of a wound care imaging and diagnosis system.

FIG. 2A shows examples of in vitro results using the wound care imaging and diagnosis system.

FIG. 2B shows clinical results of the wound care imaging and diagnosis system and its comparison with the results of some existing device and normal colored images.

FIG. 3 shows a flow chart of an example process of fluorescence imaging of wounds.

FIGS. 4A-4B show examples of UV light sources.

FIGS. 5A-5C show comparison of a normal colored image, unenhanced and enhanced fluorescent images of a wound.

FIGS. 6A-6B show examples of a user interface on a mobile device.

Like reference numbers and designations in the various drawings indicate like elements.

### DETAILED DESCRIPTION

Studies have shown that certain bacteria in wounds can produce fluorescence. Some techniques perform fluorescence imaging using a dedicated imaging device that requires a filter in front of a camera of the imaging device. The filter only allows certain colors to pass the filter and be captured by the camera. Because the resulting fluorescent image is limited to the certain colors, correctly identify bacterial infection in the wounds can be challenging. Sometimes, false detections, including false positives and/or false negatives, can be identified, even with a human expert in wound care. The reliance on filtering renders these fluorescence imaging systems extremely sensitive to ambient light. The fluorescent signals captured by the camera can be drown out by the ambient light, in which case the resulting image can be washed out. For this reason, conventional systems can only perform fluorescence imaging in a dark environment, e.g., in a dark room or with a dark drape, to obtain better fluorescent signals.

The systems and techniques described in this specification can perform fluorescence imaging by attaching an ultraviolet (UV) light source to a mobile device, without requiring an additional dedicated imaging device or filters. Specifically, the systems and techniques describe a novel, smartphone-based full visible spectrum fluorescence imaging system developed for non-invasive bacterial assessment at the point-of-care and the imaging system can perform fluorescence imaging in an ambient light environment.

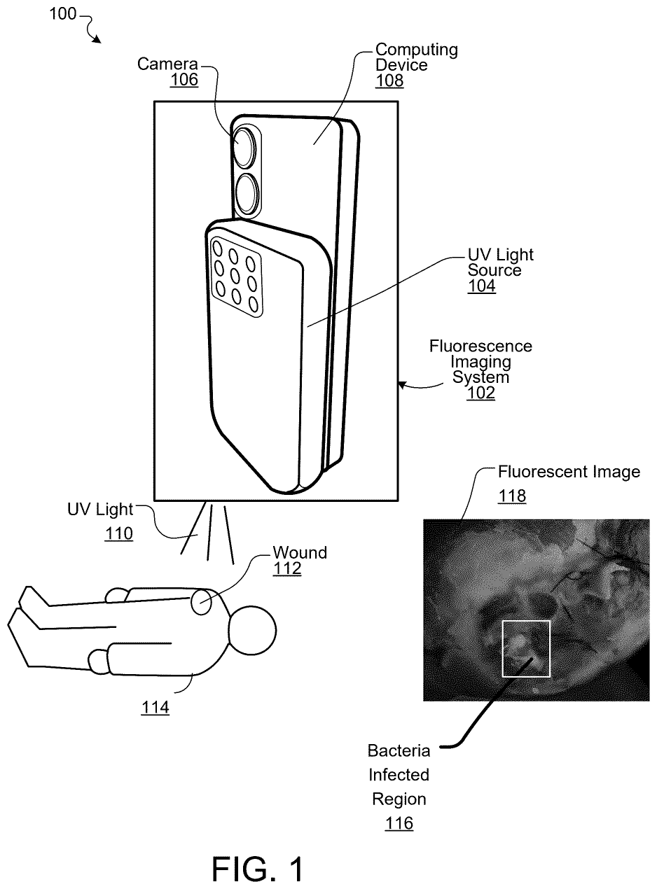

FIG. 1 is an example environment 100 of a wound care imaging and diagnosis system. The example environment 100 includes a fluorescence imaging system 102. The system 102 includes a computing device 108, one or more camera(s) 106, and an ultraviolet (UV) light source 104. The computing device 108 can be a mobile device, such as a smartphone, a laptop, or a tablet. The computing device 108 can include one or more cameras 106 that can capture an image. The camera(s) 106 can be the Red Green Blue (RGB) cameras available on a mobile device. In some implementations, the UV light source 104 can be physically attached to the computing device 108, and the UV light source 104 and the computing device 108 can be operated and moved together. Therefore, the system 102 can perform fluorescence imaging using a mobile device that a user may already have and the attached the ultraviolet (UV) light source 104, without requiring an additional dedicated imaging device.

Studies have shown that certain bacteria can produce fluorescence under the UV light. For example, some bacteria can produce red fluorescence and some bacteria can produce cyan fluorescence under the UV light. The UV light source 104 includes one or more UV light emitting diodes (LEDs) that emit UV light. The UV light source can be configured to illuminate UV light to a region of a human body, e.g., wounds, at a predetermined wavelength. Thus, the system can illuminate the wound using UV light from the UV light source 104 to detect bacterial presence in the wound.

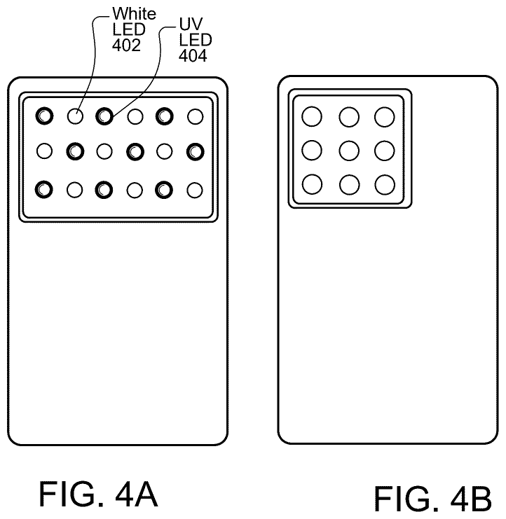

FIGS. 4A-4B are examples of UV light sources. In some implementations, the UV light source 104 can include both UV LEDs and white LEDs. The white LEDs can emit white light. The system can use the white light to take a natural color image. In some implementations, the UV light source can arrange the UV LEDs and the white LEDs in a grid, e.g., having one UV LED next to one white LED. FIG. 4A shows one example UV light source that has a three-by-six grid of UV LEDs and white LEDs. FIG. 4B shows another example UV light source that has a three-by-three grid of UV LEDs and white LEDs, e.g., four UV LEDs and five white LEDs. The UV light source design in FIG. 4B is more compact than the design in FIG. 4A and has less LEDs.

The UV light source 104 can communicate with the computing device 108 through a wired or wireless network. For example, the UV light source 104 can communicate with the computing device 108 through a Bluetooth wireless network. Other types of wired or wireless network are also possible. The computing device 108 can be configured to turn on and/or off the UV light source 104, e.g., through an application (APP). The APP can be a mobile control and imaging software. For example, the system 102 can install an APP on a smartphone. A user can control the UV light and can take a picture of the wound using the APP. When the UV light is on, the system can capture a fluorescent image. When the UV light is off, the system can capture a natural color image.

The system 102 can emit UV light 110 to a region of a human body, e.g., a wound region 112 of a human body 114, e.g., a patient. The UV light can excite fluorescent substances, such as those found in certain bacteria within the wound, causing the emission of visible light from these fluorescent substances. This secondary emitted visible light, e.g., fluorescent signals, can be captured by the camera(s) 106. The device 108 can process the fluorescent signals and can generate a fluorescent image 118 of the wound region 112. For example, the fluorescent image 118 exhibits fluorescence color in the bacteria infected region 116, e.g., areas pointed by the arrows. The device 108 can display the fluorescent image 118 on a screen of the device 108. For example, the system 102 can show the fluorescent image 118 on a user interface displayed on a screen of the mobile device 108.

Some techniques can perform fluorescence imaging using a dedicated imaging device that requires a filter in front of a camera of the imaging device. The filter may only allow certain colors to pass the filter and be captured by the camera. For example, some systems use an optical filter in front of a camera of the system so that only distinctive colors of red, green and cyan can pass through the optical filter and are captured by the camera. Because the resulting fluorescent image is limited to certain colors, the ability to interpret the resulting fluorescent image can be limited by the available colors. Thus, correctly identify bacterial infection in the wounds can be challenging. Sometimes, false detections, including false positives and/or false negatives, can be identified, even with a human expert in wound care.

The UV light source 104 can be configured to use UV light 110 to illuminate a region 112 of a human body 114 at a predetermined wavelength. A camera can have sensitivity to a wavelength within a receptive range of the camera, e.g., between 400 nm to 700 nm or between 400 nm to 900 nm, which parallels the reception band of the human eyes. In some implementations, the predetermined wavelength can be below a receptive range of the camera(s) 106. For example, the predetermined wavelength of the UV light 110 can be below 400 nm. In some implementations, to excite the fluorophores in the wound region 112, the system 102 can use UV light at the wavelength of 365 nm. The UV light with shorter wavelength (e.g., below 400 nm) can excite the fluorophores to generate fluorescent signals within the wavelength of visible light, which, once emitted, can be detected by the camera(s) 106 to form an image. The excitation light itself (e.g., UV light at 365 nm) may not be detected by the camera(s) 106 because such wavelength is outside of the receptive range of the camera(s). Thus, the excitation light can be inherently filtered out from the camera(s), without, for example, using a filter in front of the camera(s), and the excitation light may not interfere with the fluorescence signals captured by the camera(s).

Therefore, rather than using a few colors which may result in saturation issues, the systems and techniques in this specification can obtain full visible spectrum fluorescence imaging, thereby providing higher color resolution and improved image quality for human or automatic diagnosis of bacteria presence in wounds. Furthermore, because the system 102 may not require a filter, the system 102 can operate under an ambient light environment.

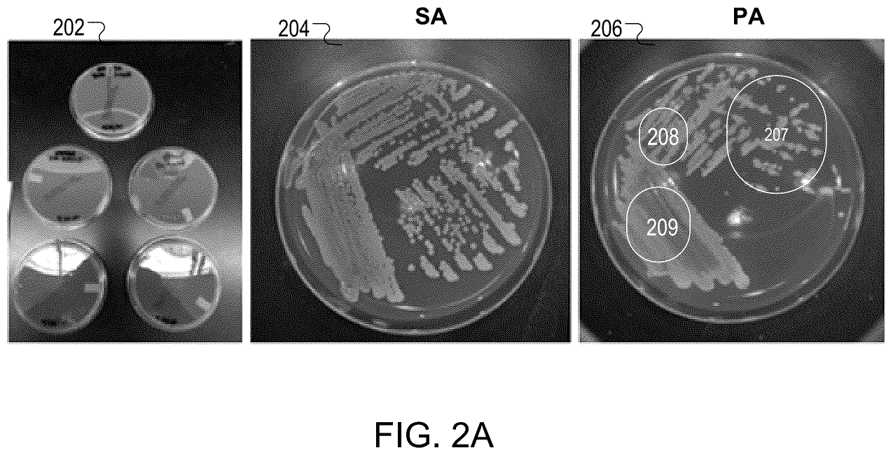

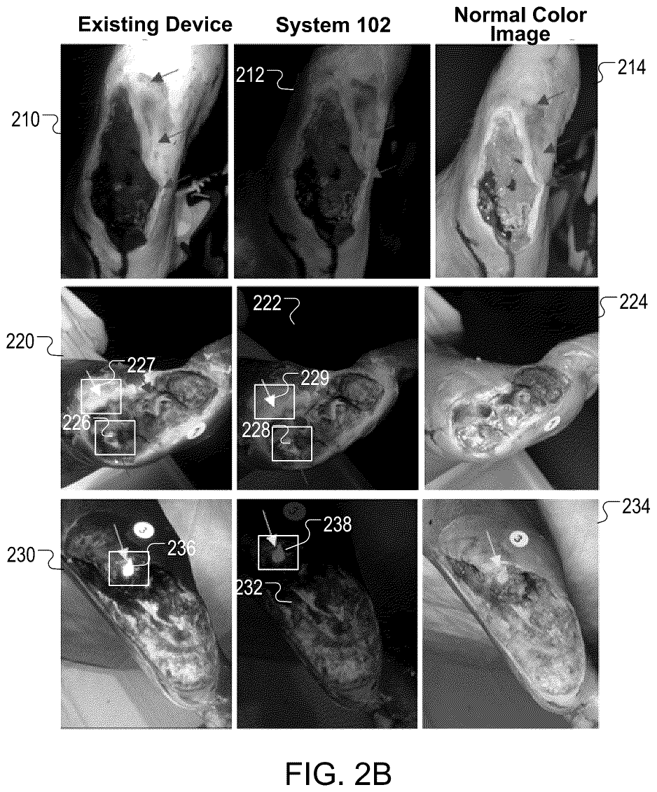

FIG. 2A show examples of in vitro results using the wound care imaging and diagnosis system. To test the performance of the system 102, two bacterial species commonly found in wound cultures, e.g., P. aeruginosa (PA) and S. aureus (SA), were prepared in vitro 202. In some cases, SA can produce red fluorescence in the image 204, whereas PA can produce both cyan fluorescence (e.g., in region 207) and red fluorescence (e.g., in regions 208 and 209) in the image 206. Such appearances result from the fluorescence of the porphyrins and pyoverdines, which are produced during the bacterial growth, under UV light.

FIG. 2B shows comparison of examples of clinical results of the wound care imaging and diagnosis system with the results from existing devices as well as colored images from the range of visible wavelengths. The middle column shows results from an initial clinical study with thirteen patients with bacterial presence (ground truth) confirmed via culture analysis. The left column shows results from an existing fluorescence imaging device. The right column shows color images from the range of visible wavelengths. Because all human tissues can include fluorophores, human tissues can generate fluorescence signals under UV light. However, the bacterial presence in the tissue can generate more intense emissions of fluorescence signals under UV light than the normal issues without bacterial presence.

The clinical results demonstrate that, while both the described system 102 and existing device can produce similar color patterns for the bacterial regions (shown with red arrows in FIG. 2B), the described system 102 can more advantageously leverage the entire visible fluorescent spectrum, thereby rendering more accurate and fulsome diagnostic information. For example, system 102 can differentiate tendon and callus from Pseudomonas growth, as illustrated by the distinct fluorescence from the bacterial region, which is absent from normal (i.e., non-bacterial) regions. In comparison, the existing system can have difficulty to differentiate tendon and callus from Pseudomonas growth, or granulation tissue from blood, as illustrated by yellow and white arrows, and the first row in FIG. 2B. In other words, the disclosed system 102 can generate fluorescence images more specifically revealing the presence of bacterial growth.

For example, the left column shows images 210, 220, and 230 captured using the existing device. The images 210, 220, and 230 mainly shows green and red color because the existing device uses a filter that only allows distinctive colors of green, red, and cyan to pass through. A user using the existing device may be instructed to look for red color and bright green color that may correspond to bacterial infected regions.

The middle column shows images 212, 222 and 232 generated using the described system 102 that uses the full visible spectrum and the resulting fluorescence images can more specifically reveal the presence of bacterial growth. For example, the region 228 at the bottom in image 222 is a region with bacteria according to culture analysis. The image 222 generated with the described system 102 exhibits cyan color and correctly identifies a bacteria infected region 228. The image 220 generated with the existing device exhibits light green color (rather than bright green) in region 226, which resembles the color of a normal tissue. Thus, a reader of the image 220 may incorrectly identify the region 226 as normal tissue, resulting in a false negative.

As another example, the region 227 in image 220 is a region without bacteria (e.g., dead skin) according to culture analysis. The image 220 shows a bright green color in this region 227. Although the region 227 is not fluorescent, because of the limitation of the design of the existing device (e.g., using the filter in front of the camera), the region 227 can exhibit a bright appearance of bright. Thus, the reader of the image 220 may incorrectly identify the region 227 as a bacteria infected region, resulting in a false positive (FP). In some implementations, a physician may need to inspect both the fluorescent image 220 and the normal color image 224 before the physician can determine that the region 227 is colorless and corresponds to a non-bacteria region. Then, the physician can rule out the FP. In contrast, the image 222 shows white color in the corresponding region 229. Thus, a reader of the image 222 can correctly identify the region 229 as a non-bacteria region. Thus, using the system 102, distinguishing bacteria infected regions from non-bacteria infected regions can be easier. A physician using the system 102 may not need to review both the fluorescent image 222 and the normal color image 224, saving the physician's time in making a diagnostic decision.

As another example, the region 236 in image 230 shows a region corresponding to another potential false positive found when using the existing device. Because the region 236 exhibits a bright appearance, a physician may determine sufficient fluorescence signals are present. However, this region corresponds to tendon, and the region has a bright appearance because bones can produce strong signals in the existing system due to the filter. The image 232 generated using the described system 102 may be free from the deceptive appearance. The region 238 in the image 232 shows a natural appearance, similar to other regions identified as not having bacteria infection. Therefore, because the described system 102 does not have such a color filter to select particular colors, the system 102 can have better color resolution and a wider dynamic range.

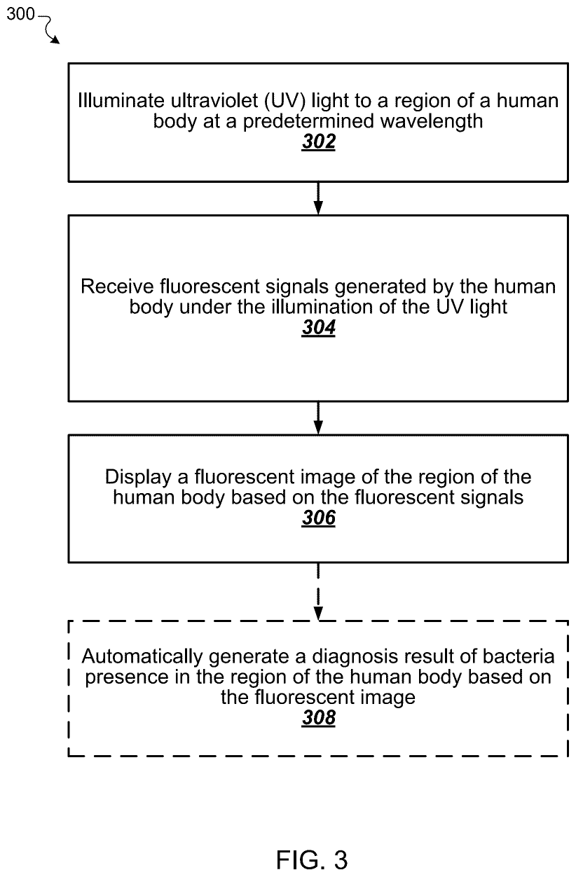

FIG. 3 shows a flow chart of an example process 300 of fluorescence imaging of wounds. The process 300 can be performed by one or more computer systems, for example, a server (e.g., a cloud computer that are located remotely from the computing device 108 and are connected with the computing device through a network), a portable electronic device (e.g., the computing device 108, the camera 105, or the UV light source 104), or a combination of these. For example, when there is no network connection, the portable electronic device can perform the process 300 locally. When there is network connection, some steps of the process 300 (e.g., performing an analysis on the fluorescent image using a neural network model or searching a database for similar images) can be performed in the cloud. In some implementations, some or all of the process 300 can be performed by the fluorescence imaging system 102.

The system uses ultraviolet (UV) light to illuminate a region of a human body at a predetermined wavelength (302). The system includes a UV light source, and the UV light source is configured to illuminate the region of the human body at the predetermined wavelength.

The region of the human body can have one or more disorders, such as a skin dermatology condition. Examples of the disorders include wound, abnormal pigmentation, such as melasma and vitiligo, bacteria infection, corneal abrasion, porphyria, scabies, head lice, skin fungus infections, skin imperfections such as acne, aging skin and milia, and tumors. The fluorescent image generated by the system can depict or characterize disorder presence in the region of the human body. In some implementations, the disorder is not limited to human body and the process 300 can be applied to fluorescent imaging of a target region of an animal (such as a pet) or any living organisms. In some implementations, the region of the human body can include a wound, and a fluorescent image generated by the system can be used to detect bacteria presence in the wound.

In some implementations, the predetermined wavelength of the UV light can be below a receptive range of the one or more cameras, e.g., shorter than 400 nanometers (nm). By using a wavelength outside the receptive range of the camera(s), the camera can block the out-of-band excitation UV light, without a need for a filter. In some implementations, the predetermined wavelength can be around 365 nm, for example, at about 370 nm, or about 360 nm. In these implementations, the predetermined wavelength can be centered at a center wavelength λ₀ with a range of wavelength (Δλ). For example, a half-strength range is the range of wavelength where the amplitude of the signal starts to fall below a threshold, e.g., 30% or 50%. Examples of the center wavelength are below 380 nm. The range of wavelength (Δλ) can be less than 20 nm, for example, 5 nm, 10 nm, or 12 nm.

The system can receive fluorescent signals emitted by the human body in response to UV illumination (304). The system includes a computing device, and the UV light source can be physically attached to the computing device. The UV light source can communicate with the computing device through a wired or wireless network. The computing device can be configured to turn on and/or off the UV light source, e.g., through an application (APP).

The computing device can include one or more cameras. The one or more cameras can be configured to receive fluorescent signals emitted from the region of the human body when illuminated with UV light. In some implementations, the system can capture the fluorescent signals emitted from the region of the human body when illuminated by UV light even in an ambient light environment. Sometimes, a physician may need to take both a color image without UV light and a fluorescent image with the UV light. Some existing devices may require performing fluorescence imaging in a dark environment, and then taking the color Image with the lights turned on. Such sequence of operations can be inconvenient and time consuming. The present system 102 can obtain images without requiring a dark environment or instant. The system can easily control the lighting from the computing device, e.g., a smart phone. The system can turn on the UV light source to obtain the fluorescent image and can quickly turn on the white light to obtain the normal color image, or can take the two images in a reversed order. Thus, the color image and the fluorescent image can be well aligned with each other (e.g., co-registered). The good alignment of the two images is beneficial for future reference, e.g., when a physician inspects a particular area of concern and compares the fluorescent image to the color image, to determine whether or not the area of concern is a positive bacteria site.

The system displays a fluorescent image of the region of the human body based on the fluorescent signals (306). The computing device can include a screen, and the screen can be configured to display the fluorescent image of the region of the human body based on the fluorescent signals.

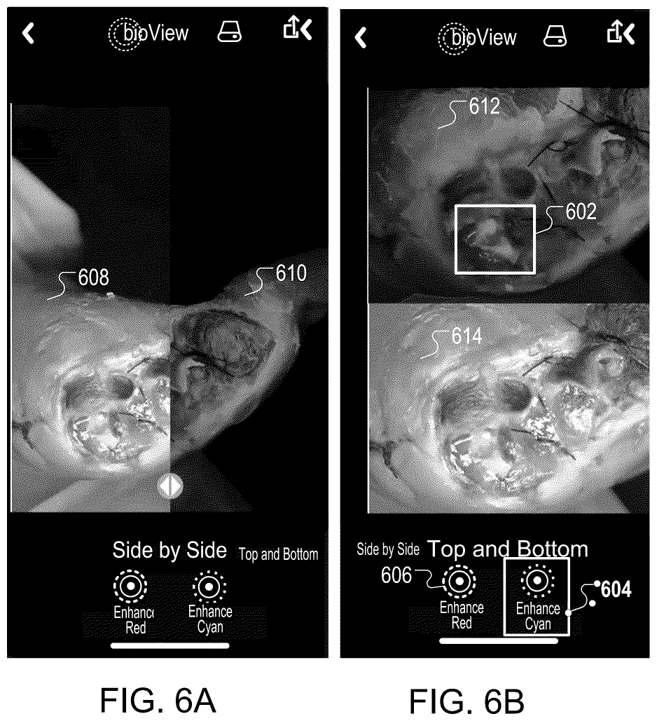

In some implementations, the system can perform post processing of the fluorescent image. In some implementations, the system can generate an enhanced fluorescent image of the region of the human body based on the fluorescent image, and the system can display the enhanced fluorescent image on the screen. In some implementations, the system can divide the colors in the fluorescent image into Hue values and Saturation values. Based on the Hue value, which corresponds to color, the system can perform the enhancement. For example, the system can generate an enhanced fluorescent image that enhances a color of interest, e.g., Red or Cyan. For example, the upper portion of FIG. 6B shows an enhanced fluorescent image that enhances Cyan color at least in the region 602, if the user clicks on the “Enhance Cyan” button 604. If the user clicks on the “Enhance Red” button 606, the system can display an enhanced fluorescent image that enhances Red color (not shown in FIG. 6B). In some implementations, the system can decompose the fluorescent image into different wavelength components. Based on these varied wavelength signals, the system can conduct fluorescence spectroscopy and carry out advanced bacterial load analysis.

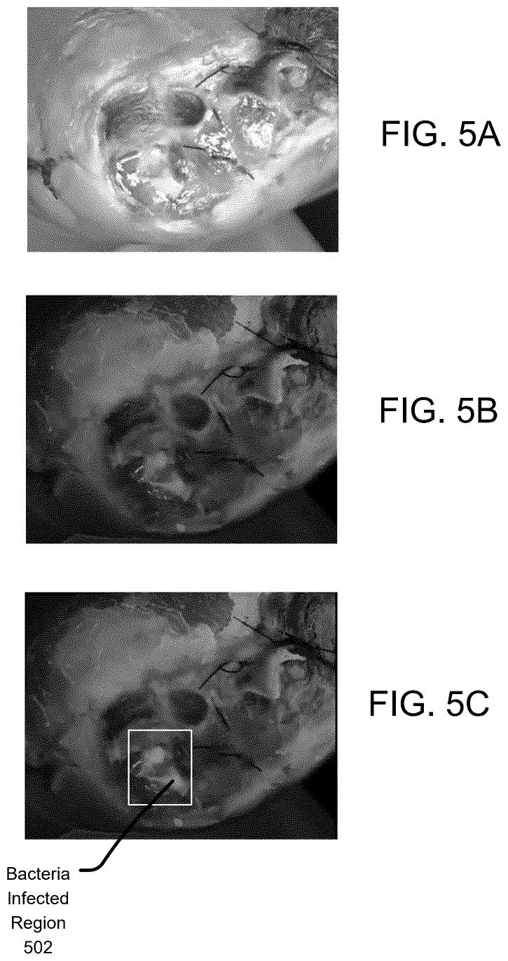

FIGS. 5A-5C show comparison of a colored image (FIG. 5A), unenhanced (FIG. 5B) and enhanced (FIG. 5C) fluorescent images of a wound. FIG. 5C shows a fluorescent image with enhanced cyan color. The system can detect the cyan color in the fluorescent image. Then the system can increase the intensity of the pixels corresponding to the cyan color. Because the cyan color in the fluorescent image may correspond to bacteria infected region (e.g., region 502), performing diagnosis of bacteria presence in the wound using the enhanced fluorescent image can be easier.

In some implementations, the system can capture a natural color image of the region of the human body using the one or more cameras. The natural color image is captured without the UV light. For example, the system can capture the natural image with the camera's LED, the UV light source's white LED, or no LED if there is sufficient ambient light. The system can display both the natural color image and the fluorescent image on the screen. In some implementations, the system can display the natural color image, the fluorescent image, the enhanced fluorescent image, or a combination of these, on the screen. FIGS. 6A-6B show examples of a user interface on a mobile device.

FIG. 6A shows a side-by-side view of a natural color image and a fluorescent image. The user interface includes a slider that allows a user to slide between the natural color image 608 on the left and the fluorescent image 610 on the right that overlays on top of each other. The buttons at the bottom can be activated to show red enhanced fluorescent image or cyan enhanced fluorescent image. FIG. 6B shows a top and bottom view of a natural color image 614 at the bottom and a cyan enhanced fluorescent image 612 at the top.

In some implementations, the system can automatically generate a diagnostic result of bacteria presence in the region of the human body based on the fluorescent image (308). This system, employing advanced machine learning and computer vision techniques, can interpret the variations in color and intensity in the fluorescent image, denoting different species of bacteria or the formation of biofilms. The unique fluorescence patterns of different bacterial species or biofilms can be classified, and the learning algorithm can be trained on these patterns. Moreover, certain bacteria are known to produce biofilms, which are organized communities of bacteria that are embedded in a self-produced matrix. Such biofilms, which could contribute to chronic infections, might have unique autofluorescence patterns that could be captured and identified by the system.

In some implementations, the system can analyze the fluorescent image alongside a color image. By doing this analysis, the system can provide more context to the fluorescent image, such as the location and size of the wound, or its stage of healing, while also giving detailed information about bacterial presence. This comprehensive analysis could offer a more overarching perspective on wound care.

In some implementations, the diagnostic result can include a score indicating likelihood that there are bacteria in the wound. For example, the system can generate a score of 85%, indicating that the likelihood that there are bacteria in the wound is 85%. In some implementations, the diagnostic result can include data indicating one or more regions in the wound that likely have bacteria. In some implementations, the system can include a segmentation machine learning (ML) model. Examples of the segmentation machine learning model include a deep learning model, specifically a convolutional neural network (CNN) designed for image segmentation tasks, such as a Mask R-CNN (Region-based Convolutional Neural Networks) model. Leveraging the deep learning model, the system can assess a complex wound with multiple types of bacteria detected through an advanced fluorescence imaging setup. In such scenarios, the system incorporates segmentation model (Mask R-CNN) that accurately identifies the precise regions within the wound where each bacterial species resides. Moreover, in instances with biofilm presence, the Mask R-CNN model can not only discern the specific wound areas occupied by the biofilm, but also characterize the shape, texture, and potential severity of the biofilm. These detailed identifications and assessments collectively aid in developing an accurate, targeted treatment strategy, thereby enhancing the efficiency and effectiveness of wound care management.

In some implementations, the system can provide the fluorescent image as an input to an AI-based diagnostic engine, and the system can automatically generate the diagnostic result via the application of advanced machine learning algorithms. The machine learning algorithm can be previously trained to take an input fluorescent image (or in addition, the natural color image) and generate, as an output, a comprehensive wound diagnosis of the input fluorescent image. This ML algorithm performs classification of the bacterial after the region containing bacterial has been segmented, for example, using the earlier described segmentation ML model.

To further increase accuracy, the machine learning algorithm could incorporate a Convolutional Neural Network (CNN) model. The CNN would be capable of effectively learning spatial hierarchies from the images, thus allowing for a deeper and more precise analysis of the wound, including the recognition and quantification of specific bacterial species through their fluorescent patterns.

Our CNN model incorporates the principle of deep learning, with network depth that can vary according to the complexity of the wound and the diversity of bacteria present. In one example, a hierarchical structure including 5-7 layers can offer the most balanced performance between computational efficiency and accuracy. This structure enables the system to learn complex patterns and variations in fluorescence signals unique to different bacteria. Significantly, the implementations can include algorithmic modifications to tune the CNN model specifically for wound image analysis. First, the initial layers of the network can be implemented to focus on capturing low-level wound features such as color, shape, texture, and overall wound structure. This modification can enhance the model's power to differentiate between the wound and the surrounding normal tissue. The middle layers of the network can concentrate on recognizing more specific features such as bacterial fluorescent patterns which are salient identifiers of specific bacteria types. Lastly, the final layers of the network can tie these patterns with stored information of known bacteria types and their respective fluorescence signals to generate the final classification result. Such a hierarchically structured CNN model capitalizes on the pattern recognition power of deep learning to enhance the effectiveness and precision of wound diagnosis. The hierarchically structured CNN model thus progresses from broader, simpler wound features to more nuanced, specific bacterial identifiers, thereby enabling a comprehensive understanding of the wound's bacterial environment.

In another implementation, the system could employ a combination of CNN and Recurrent Neural Network (RNN) models. RNNs are adept at processing sequential data, allowing the system to track changes in wound appearance over time and potentially predicting healing trajectories based on current and past images.

Additionally, a Generative Adversarial Network (GAN) could be used not only to enhance the interpretability of the fluorescent images but also to simulate possible wound evolution scenarios under different treatment strategies. This approach could provide healthcare providers with a unique and valuable tool for personalized patient treatment planning.

Employing GANs in exemplary implementations can accomplish multiple objectives. In one salient example, the GAN model can be trained to enhance the interpretability of the fluorescent images. This process involves training the model on large amounts of fluorescence image data; the “generative” part of the model learns to create new, synthetic images, while the “adversarial” part critically evaluates these generated images for authenticity. Through iterative learning, the GAN model can eventually generate high-quality images that leverage noise and detail distribution from the real data, improving image interpretability for human viewers. In some implementations, the GAN model is a powerful tool for prediction and scenario planning. By leveraging historical wound evolution data for patients with similar wound characteristics and bacterial profiles, in some implementations, the GAN model can be trained to generate likely wound evolution scenarios under different treatment strategies. For instance, for a wound with observed bacterial infection, the GAN model can highlight potential variations in the state of the wound while the wound undergoes interventions such as antibiotic therapy, surgical debridement, or alternate wound dressing techniques. These simulated evolutions give healthcare providers a visual, comparative tool to evaluate the effectiveness and potential impact of diverse treatment strategies. Significantly, in predicting wound evolution, the GAN model can generate sequential, temporal data, enabling a time-lapse view into the wound's healing process. This feature provides an intuitive, visual tool for tracking the progress of wound recovery or the worsening of wound infection, and can significantly aid in proactive and timely decision-making.

Leveraging unsupervised machine learning algorithms such as clustering could provide another layer of diagnostic power by grouping regions of the image according to common fluorescence characteristics, potentially unveiling hidden patterns related to bacteria behavior, interactions, and growth.

Through a smart combination of these advanced AI techniques, the proposed system would offer unprecedented levels of accuracy and precision in wound diagnosis and treatment planning using fluorescence imaging.

In some implementations, the system can generate a quantitative diagnostic result of bacteria presence (e.g., a level of bacterial infection) in the wound based on the fluorescent image and one or more parameters of the UV light source and the one or more cameras. Besides qualitative measurements, the system can allow a more quantitative way to control the variables, including parameters of the ambient light sensor, camera exposure and other parameters of the camera, strength of the UV, and other parameters of the system. By controlling these variables, the system can quantify the signal strength of the emitted UV light. In one illustrative example, the system can adjust the intensity of the illuminating UV light, thereby managing potential photobleaching and saturation issues that may influence image clarity. Although most of the wound has some level of bacteria, by controlling the strength of the UV light, the system can generate better fluorescent images that can be used to determine whether a region of interest in the image corresponds to a bacteria region with bacterial infection.

For example, the system can include one or more sensors, including an ambient light sensor, a temperature sensor, and a distance sensor, to determine one or more environmental parameters that can affect image quality and UV light consistency. The ambient light sensor measures the intensity of the surrounding light. The temperature sensor records the ambient temperature, and the distance sensor gauges the proximity of the imaging device to the wound site. Based on the data acquired from these sensors, the system can dynamically adjust the parameters of the camera device (such as exposure and ISO) and the parameters of the UV light source (such as its strength). These adjustments ensure that the UV light maintains a consistent signal strength within the region of interest (ROI). By achieving consistent UV illumination, the system ensures that consecutive images of the wound are comparable, allowing for accurate assessment of bacterial load changes over time. For instance, if the ambient light sensor detects an increase in surrounding light brightness, the system may reduce the camera's exposure or adjust the UV light strength to counterbalance the effect and maintain consistent illumination. Similarly, changes in temperature or distance from the wound can prompt adjustments to maintain image quality and consistency. This precise control mechanism enables the device to produce reliable and comparable images for successive bacterial load assessments, significantly improving the accuracy and effectiveness of wound care management.

In some implementations, fluorescence imaging system can be equipped with fluorescence signal modulation functions for enhanced and precise detection of bacterial loads in wounds. The modulation of fluorescence signals exploits the distinctly characteristic properties of the fluorescence by different bacterial species, such as unique emission spectra and intensity variations logged over time. This modulation can be achieved by incorporating digital communication methodologies to control the UV light source that instigates the fluorescence, or by adjusting the exposure parameters, gain controls, and signal processing algorithms of the imaging camera. For instance, the UV light source can be modulated using pulse-width modulation to variably control the light duration when illuminating the wound area. The modulation can also include frequency modulation to adjust the rate at which the UV light illuminates the wound area. Additionally or alternatively, the modulation can include pulse-intensity modulation when neighboring pulses for UV illumination are modulated in intensity. These techniques can reduce image noise and enhance the contrast between autofluorescent bacterial signals and surrounding tissue fluorescence. Likewise, the camera's exposure settings, gain values, and signal processing routes can also be modulated for further enhancement of the imaging contrast. These modulation techniques can give rise to a new and improved level of image clarity, which in turn enables improved discrimination between different bacterial species within complex wounds that harbor multiple microbiota. In scenarios involving biofilms, the capacity to modulate the fluorescence signals becomes crucial in identifying the structure, composition, and severity levels of biofilms. The accurate detection and differentiation of biofilms can then influence the establishment of precise treatment strategies.

In some implementations, after generating the fluorescent image of a wound, the system can search a database of previously captured images to retrieve one or more images that are similar to the generated fluorescent image. The system displays the generated fluorescent image with the one or more retrieved images (and its diagnosis report) on a screen. The diagnostic result of the one or more retrieved images can be helpful when performing diagnosis of the wound using the fluorescent image. This database can be an integral part of the system. Additionally or alternatively, the database can be an externally located cloud-based repository, presenting the advantage of continually updated and diversified data. The system can auto-search this database to retrieve one or more images that bear semblance to the freshly generated fluorescent image of the wound. This matching process could be powered by advanced algorithms, traditionally employed in image recognition tasks. For example, a potential use case could be a wound exhibiting bacterial load with unusual fluorescence patterns so that relevant images can be retrieved using the advanced algorithms. By showcasing similar cases and their outcomes, the system can provide valuable context and precedent for diagnosis. Additionally, wounds healing over time might exhibit evolving fluorescent characteristics. A timeline of images retrieved from the same patient's history could enable medical professionals to visually trace the progress of healing. Lastly, in situations where wounds display multiple bacterial types or advanced stages of infection, the comparison to precedent images could highlight nuanced inferences. These comparisons might be critical to formulate an optimal treatment strategy.

The order of steps in the process 300 described above is illustrative only, and the process 300 can be performed in different orders. In some implementations, the process 300 can include additional steps, fewer steps, or some of the steps can be divided into multiple steps.

Embodiments of the subject matter and the operations described in this specification can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer programs, i.e., one or more modules of computer program instructions, encoded on computer storage medium for execution by, or to control the operation of, data processing apparatus. Alternatively, or in addition, the program instructions can be encoded on an artificially-generated propagated signal, e.g., a machine-generated electrical, optical, or electromagnetic signal, which is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium can also be, or be included in, one or more separate physical components or media (e.g., multiple CDs, disks, or other storage devices).

The operations described in this specification can be implemented as operations performed by a data processing apparatus on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

This document refers to a service apparatus. As used herein, a service apparatus is one or more data processing apparatus that perform operations to facilitate the distribution of content over a network. The service apparatus is depicted as a single block in block diagrams. However, while the service apparatus could be a single device or single set of devices, this disclosure contemplates that the service apparatus could also be a group of devices, or even multiple different systems that communicate in order to provide various content to client devices. For example, the service apparatus could encompass one or more of a search system, a video streaming service, an audio streaming service, an email service, a navigation service, an advertising service, a gaming service, or any other service.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random-access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and typically interact through a communication network. The relationship of client and server arises by virtue of computer programs running on the respective computers and having a client-server relationship to each other. In some embodiments, a server transmits data (e.g., an HTML page) to a client device (e.g., for purposes of displaying data to and receiving user input from a user interacting with the client device). Data generated at the client device (e.g., a result of the user interaction) can be received from the client device at the server.

While this specification contains many specific implementation details, these should not be construed as limitations on the scope of any inventions or of what may be claimed, but rather as descriptions of features specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

1. A system, comprising:
an ultraviolet (UV) light source activatable to illuminate a region of a human body using UV light at a predetermined wavelength; and
a computing device to which the UV light source is attachable, wherein the computing device is arranged in communication with the UV light source and capable of activating the UV light source, the computing device comprising:
a camera device configured to detect fluorescent signals emitted from the region of the human body in response to illumination using the UV light at the predetermined wavelength that is below a band of human visual perception,
a screen,
a memory device, and
one or more processors coupled to the camera device, the screen, and the memory device, wherein the one or more processors are configured to:
obtain one or more environmental parameters capable of affecting the fluorescent signals as detected by the camera device;
based on the one or more environmental parameters, adjust an operation on at least one of: the camera device, and the UV light source, to compensate for varied detection of the fluorescent signals caused by the one or more environmental parameters; and
based on the fluorescent signals detected by the camera device, generate a fluorescent image for displaying on the screen.

2. (canceled)

3. The system of claim 1, wherein, once activated, the UV light source illuminates the region of the human body under ambient light.

4. (canceled)

5. The system of claim 1, wherein the predetermined wavelength is shorter than 400 nanometers (nm).

6. The system of claim 5, wherein the predetermined wavelength is about 365 nm.

7. The system of claim 5, wherein the predetermined wavelength is characterized by a center wavelength along with a width of wavelength, wherein the center wavelength is shorter than 380 nm, and wherein the width of wavelength is less than 20 nm.

8. The system of claim 1, wherein the UV light is modulated when illuminating the region of the human body.

9. The system of claim 8, wherein the UV light is modulated according to at least one of: a pulse width modulation, a pulse intensity modulation, or a frequency modulation.

10. The system of claim 1, wherein the one or more processors are configured to analyze the fluorescent image by:
segmenting at least one region in the fluorescent image with likely bacterial presence using a machine learning model to discern characteristic features of bacterial induced fluorescence.

11. The system of claim 1, wherein the one or more processors are configured to:
process sequential fluorescent images of the region of the human body using a machine learning model;
track a change in the sequential fluorescent images of the region of the human body; and
predict a healing trajectory of the region of the human body.

12. The system of claim 1, wherein the one or more processors of the computing device are configured to enhance the fluorescent image.

13. The system of claim 12, wherein the one or more processors of the computing device are configured to:
generate Hue values of the fluorescent image; and
generate the enhanced fluorescent image based on the Hue values of the fluorescent image.

14. The system of claim 12, wherein the one or more processors of the computing device are configured to:
process the fluorescent image using a Generative Adversarial Network (GAN) trained on a dataset of fluorescent images to generate the enhanced fluorescent image.

15. The system of claim 1, wherein the one or more processors of the computing device are configured to process the fluorescent image using a Generative Adversarial Network (GAN) to generate a prediction of at least one future state of the region of the human body under at least one treatment scenario.

16. The system of claim 15, wherein the one or more processors of the computing device are configured to:
provide, based on the at least one predicted future state, a visual simulation of how the region of the human body evolves under the least one treatment scenario.

17. The system of claim 1, wherein the fluorescent image is a fluorescent image of a wound area of the human body.

18. A method performed by one or more computer processors coupled to an ultraviolet (UV) light source and a camera device, the method comprising:
obtaining, from at least one sensor, one or more environmental parameters capable of affecting a camera device's detection of fluorescent signals being emitted from a region of a human body in response to illumination from the UV light source;
based on the one or more environmental parameters, adjusting, by the one or more computer processors, an operation on at least one of: the camera device, and the UV light source, to compensate for varied detection of the fluorescent signals caused by the one or more environmental parameters;
receiving, from the camera device, fluorescent signals emitted from the region of the human body in response to the UV light source being activated to transmit UV light at a predetermined wavelength that is below a band of human visual perception; and
based on the fluorescent signals detected by the camera device, generating, by the one or more computer processors, a fluorescent image of the region of the human body.

19-35. (canceled)

36. The method of claim 18, wherein the UV light source illuminates the region of the human body under ambient light.

37. The method of claim 18, wherein the one or more environmental parameters are obtained from at least one of: an ambient light sensor, a temperature sensor, or a distance sensor.

38. The method of claim 37, wherein the fluorescent signals are detected by the camera device when the ambient light sensor detects an ambient signal level below a threshold level.

39. The method of claim 18, wherein the operation pertains to at least one of: an exposure of the camera device, an ISO setting of the camera device, and a strength of the UV light.

40. The method of claim 18, wherein the predetermined wavelength is shorter than 400 nanometers (nm).

41. The method of claim 40, wherein the predetermined wavelength is about 365 nm.

42. The method of claim 40, wherein the predetermined wavelength is characterized by a center wavelength along with a width of wavelength, wherein the center wavelength is shorter than 380 nm, and wherein the width of wavelength is less than 20 nm.

43. The method of claim 18, further comprising: modulating the UV light when illuminating the region of the human body.

44. The method of claim 43, wherein said modulating is performed according to at least one of: a pulse width modulation, a pulse intensity modulation, or a frequency modulation.

45. The method of claim 18, further comprising:
generating consecutive florescent images based on consecutively detecting the fluorescent signals in response to repeated illumination using the UV light.

46. The method of claim 18, further comprising:
decomposing the fluorescent image into wavelength components; and
performing fluorescence spectroscopy analysis using the wavelength components of the fluorescent image.

47. A non-transitory computer-readable medium encoding instructions operable to cause a data processing apparatus to perform operations comprising:
obtaining one or more environmental parameters capable of affecting a camera device's detection of fluorescent signals being emitted from a region of a human body in response to illumination from an ultraviolet (UV) light source;
based on the one or more environmental parameters, adjusting an operation on at least one of: the camera device, and the UV light source, to compensate for varied detection of the fluorescent signals caused by the one or more environmental parameters;
receiving, from the camera device, fluorescent signals emitted from the region of the human body in response to the UV light source being activated to transmit UV light at a pre-determined wavelength that is below a band of human visual perception; and
based on the fluorescent signals detected by the camera device, generating a fluorescent image of the region of the human body.

48. The system of claim 1, wherein the one or more environmental parameters are obtained from at least one of: an ambient light sensor, a temperature sensor, or a distance sensor.

49. The system of claim 1, wherein the operation pertains to at least one of: an exposure of the camera device, an ISO setting of the camera device, and a strength of the UV light.
